(19) United States
(12) Patent Application Publication
(10) Pub. No.: US 2026/0265101 A1
(43) Pub. Date: Sep. 10, 2026

(54) Exploitation Of Wine Processing Waste By Means Of Integrated Biological Processes Of Anaerobic Digestion And Microalgae Culture

(71) Applicant: UNIVERSITA' CA FOSCARI VENEZIA, Venezia (IT)

(72) Inventors: Cristina CAVINATO, Lonato del Garda (IT); Paolina SCARPONI, Via Farnesiana 48 (IT)

(21) Appl. No.: 18/869,565

(22) PCT Filed: May 31, 2023
(86) PCT No.: PCT/IB2023/055555

§ 371 Date: Nov. 26, 2024

(30) Foreign Application Priority Data
Jun. 1, 2022 (IT) 102022000011675

(51) Int. Cl. C02F 3/34; C02F 3/00; C02F 11/04; C02F 103/32; C12F 3/06; C12N 1/12; C12R 1/89

(52) U.S. Cl. CPC C02F 3/34; C02F 3/006; C02F 11/04; C12F 3/06; C12N 1/12; C02F 2103/325; C12R 2001/89

(57) ABSTRACT

The present invention discloses a process mode of microalgal cultivation in semi-continuous on wine wastewater with no need to recirculate the microalgal biomass in the reactor, without dilution and sterilisation of the effluent applied as a growth medium. The process was experimentally carried out by semi-continuously applying, without recirculation of the biomass, the cultivation of Chlorella vulgaris on the digestate from the anaerobic co-digestion of winemaking lees and waste activated sludge. The integrated anaerobic digestion-microalgae treatment process is advantageous from a circular economy perspective as it leads to the exploitation of grape processing chain waste, eliminating the economic burden on producers and involving: i) the phyto-depuration of the effluent, ii) the production of bio-methane and iii) the production of biomass containing high value-added by-products (lipids, proteins and starch) that can be used to produce biodiesel, animal feed, nutraceuticals/pharmaceuticals and for producing bioplastics.

### FIELD OF THE INVENTION

The present invention relates to the technical field of solid organic waste disposal and transformation thereof into something useful or less harmful; in particular, it relates to a process for cultivating microalgae on anaerobic digestate of wine-growing waste.

### BACKGROUND ART

### Italian Wine-Growing Framework and Circular Economy of Winemaking Lees

Italian wine production is spread throughout the country and suffers, like all agricultural production, from the impact of the waste produced associated with the end product. Wine-growing waste refers to the solid organic waste produced during grape processing, CO₂ emissions, winemaking lees and waste water produced during the winemaking process. The greatest production of wine-growing derived waste occurs mainly in the autumn months (September-October-November) during the grape harvest, but actually, considering the different stages of wine processing, this production is indeed annual (i.e. lees produced by the wine barrel-ageing process). The winemaking process is complex and strongly linked to the product to be obtained. In general, the winemaking process, starting with the grape harvest, involves various treatment steps: pressing, fermentation, sedimentation/settling, maturation, clarification and, finally, bottling. Establishing the amount of wine waste produced is extremely complex and depends on many factors, from the production technologies applied to the size of the cellar. Typically, each hectolitre of wine produced corresponds to 196 l of waste water, 0.1 kg of waste activated sludge (WAS) and 1.6 kg of wine lees [Da Ros C, et I. J Environ Manage 2017, 203, 745-52]. In Italy, the annual wine production is 53 million quintals and the estimated production of lees is 2,140 million quintals, taking into account both the lees produced during the grape harvest and those obtained from ageing the wine in barrels. The winemaking lees in wine production play a crucial role in obtaining a quality product. The winemaking lees promote the natural detoxification of the final product from organic compounds released by the micro-organisms carrying out fermentation and play a crucial role in the interaction with the polyphenolic compounds that provide the colour and organoleptic properties of the final product (wine).

Lees, to date, do not find alternative applications, as is the case for grape marc, thus remaining a waste with a disposal and storage cost borne by producers as defined in Law 234/2016 “Disciplina organica delle coltivazione della vite e della produzione e del commercio del vino”, Official Gazette No 302 of 28 Dec. 2016, known as the “Testo Unico del vino” [Novello V. Viticoltura 2015, 61-4].

Given the need to find applications for the circular economy of winemaking lees, the exploitation thereof has been studied mainly with the purpose of recovering substances of interest, such as antioxidants and bioethanol [Cortés A, et al. Waste Manag 2019, 95, 70-7], but with still high associated costs. loannidou et al. [Bioresour Technol 2020, 307, 123093] have summarised and quantified the products of economic interest that may be obtained from lees treatment, distinguishing different treatments applicable to the two, liquid and solid, post-centrifugation fractions. However, the series of steps required and the need for solvents and enzymes make the treatment process expensive and difficult to apply [Ioannou L A, et al. J Hazard Mater 2015, 286, 343-68].

International events, such as Vinitaly and Enoforum (2013-2014), have drawn the attention on innovative applications for the treatment and exploitation of wine wastewater, showing the interest of Italian producers in research and innovation in the circular economy. This interest is closely related to the particular Italian production landscape, where wine production is mainly associated with small private family businesses (42-46%), cooperatives processing the grapes of small producers (42-46%) and only a small fraction with large private entities processing the grapes of third parties (8-16%).

The scenario shown by analysing the productions and types of companies cooperating in wine production emphasises the strong influence of small companies, which require new waste management and disposal methods that are applicable, and easy to manage, both for small wine producers and consortia as well as for large companies. There is therefore a clear need to identify alternative applications of wine waste, aimed at using waste directly on site for small and large companies, which would lead to a reduction in environmental impact by avoiding unauthorised environmental spillage, which typically occurs in sewers or watercourses, and at producing products with economic value for the companies.

### Characteristics of Wine Lees

The characterisation of the wine lees shows the presence of micro-organisms (yeasts, which have previously allowed alcoholic fermentation of the wine), polyphenols, lignin, metals, proteins, organic salts (tartrates), skins, seeds, bunches and stalks, extremely low pH (around 3), high chemical oxygen demand (COD) and high biological oxygen demand (BOD), which prevents its release into the environment as established by the law and requires pre-treatments [D. Lsg 479/2008]. From an environmental point of view, the release of lees onto the soil causes karstification of the limestone components, in an anaerobic environment it leads to the production of methane and carbon dioxide, and in an aquatic environment it leads to sterilisation of the biota. Among the various wine wastes that can be obtained from wine production, winemaking lees, or wine lees, are an interesting substrate for anaerobic digestion (AD) systems due to their high biodegradability, high organic content, year-round availability associated with barrel ageing; as it is not used in terms of circular economy perspective, this waste water represents a new and promising substrate for biogas production by eliminating the costs associated with storage and disposal that are borne by wine producers. Wine lees typically consist of 10.4% protein, 1.2% fatty acids, 5.6% ash and 20.7% tartaric acid [Ioannidou et al. Bioresour Technol 2020, 307, 123093]. Although the production of wine lees is active throughout the year, there is a considerable increase in production in the autumn period. The application of a winemaking wastewater treatment plant therefore requires system flexibility in response to waste fluctuations produced during the year.

There is little research on the application of AD processes for the treatment of lees. Few works have evaluated the application of lees, including Hungría et al. [Environ Technol (United Kingdom) 2020, 0, 1-9], Da Ros et al. [Biomass and Bioenergy 2016, 91, 150-9] and Montalvo et al. [Sustain Energy Technol Assessments 2020, 37, 100640]. In general, there is a need for a lees-WAS co-digestion to increase methane production with an applicable organic loading rate (OLR) for mesophilic AD treatment of lees of 3.2 kg_{COD}/m³ d and hydraulic retention time (HRT) of 23 days [Da Ros et al. Waste Manag 2014, 34, 2028-35]. In one year of experimentation using only white wine lees, the specific gas production (SGP) obtained was 0.38 Nm³/kg_{CODalimented} with a 65% methane composition during the steady state [Da Ros et al. Waste Manag 2014, 34, 2028-35]. The characteristics of the wine digestate obtained by Da Ros et al. [Biomass and Bioenergy 2016, 91, 150-9] shows ammonia concentration values leaving the system of 0.40±0.05 g_{N-NH4}/l and soluble COD of 1167±841 mg_{sCOD}/kg_{ww}, thus requiring pre-treatment before being released into the environment (Legislative Decree 152/06). The characterisation of metals in wine digestate shows the presence of zinc (1199 mg/kg_{dw}), copper (929 mg/kg_{dw}), lead (115 mg/kgₛₛ), chromium (48 mg kg_{dw}), nickel (26 mg/kg_{dw}), cadmium (1.6 mg/kg_{dw}) and mercury (0.4 mg/kg_{dw}). Although most of the metals identified are not in a high amount, copper and zinc show very high values associated with the type of lees that can contribute up to 70% for copper entering the AD system. The increase in heavy metals in the AD effluent may be the limiting factor for a following tertiary biological treatment.

However, the AD treatment of wine lees produces an effluent (digestate) with chemical characteristics that limit the release thereof into the environment pursuant to the Legislative Decree 152/06, thus emphasising the need for further treatment aimed at removing ammonium.

### Microalgae: Applications in Wastewater Treatment

Wastewater with high nitrogen and phosphorous concentrations, such as effluents from anaerobic digestion, require pre-treatment before being released into the environment as their discharge into the aquatic environment causes eutrophication. Eutrophication is a process wherein organic and inorganic compounds in wastewater have a fertilising effect on microalgal species; fertilisation leads to a substantial increase in the concentration of microalgal biomass causing anoxia (lack of oxygen) of the aquatic environment with substantial environmental damage. Given the environmental effect of wastewater on microalgal proliferation, the application of wastewater for the controlled cultivation of microalgae in a closed environment (photobioreactors) has two main advantages: the phyto-depuration of wastewater associated with the removal of organic and inorganic compounds, and the production of microalgal biomass rich in high-value-added by-products that can be applied to various industrial sectors, from biodiesel production to animal feed. In terms of advantages, the application of wastewater for microalgal cultivation becomes an alternative and sustainable way of exploitation, due to i) the presence of nutrients otherwise supplied through synthetic growth media and ii) the reduction of clean water use, both of which also contribute to cost reduction.

The assessment of microalgal growth on wastewater for the identification of efficient tertiary biological treatment was evaluated on both single microalgal species and on microalgal or microalgae-bacteria consortia. The application of microbiological polycultures as a tertiary treatment of wastewater shows advantages in treatment robustness and efficiency, positively affecting wastewater purification through the syntrophic effect associated with mixed cultures (as in anaerobic digestion systems) and increasing the robustness of the system in response to environmental changes and pathogen resistance. Among the many applicability studies of microalgal strains 15 in wastewater treatment, Scenedesmus obliquus, Chlorella vulgaris, Nannochloropsis salina and Chlorella pyrenoidosa, were the most studied microalgal species. [Gonçalves A L, et al. Algal Res 2017, 24, 403-15]. The accumulation of by-products (i.e. lipids, proteins and starch) leads to multiple applications of microalgal biomass extracts. To date, third-generation biofuels derive from the conversion of microalgal oils into biodiesel, thus requiring strains that can grow continuously, thus producing high concentrations of oil that can be continuously converted into biodiesel. Finally, it is also possible to exploit the starch content by fermentation into bioethanol or, due to the high protein content, it is possible to use it as a food supplement in animal feed. The amino acid component obtained from microalgae is in fact rich in essential amino acids that, as they are not synthesised by higher organisms, must be supplemented in their diet.

Among the types of wastewater most studied for application as a growth medium for microalgae cultures, the anaerobic digestion effluent (digestate) has shown great potential for use due to its abundance of ammonium and phosphorus. The problems associated with the application of this treatment system are mainly related to the need for light irradiation for mixotrophic cultures, as the cloudiness of the digestate and the presence of suspended solids prevent the penetration of light irradiation. The application of the digestate as a substrate for microalgal growth therefore requires pre-treatment prior to its use, such as filtration [Yang L, et al. Bioresour Technol 2015, 181, 54-61], settlement [Tan X, et al. Bioresour Technol 2014, 170, 538-48], sterilisation and/or ozonation [Cheng J, et al. Bioresour Technol 2016, 216, 273-9], dilution [Wang L, et al. Appl Biochem Biotechnol 2010, 162, 2324-32], centrifugation [Åkerström A M, et al. J Environ Manage 2014, 144, 118-24], up to ultraviolet treatment [Zhao Y, et al. Bioresour Technol 2015, 187, 338-45]. The increase in the amount of pre-treatment required implies an increase in system costs and this is why methods with a lower economic impact are sought.

Furthermore, in order to make the process economically feasible, it is necessary to have a continuous production of biomass combined with continuous treatment of the effluent; this excludes batch cultivation which is too expensive and with time-consuming treatment and recovery and not industrially applicable.

Scarponi P, et al. (Waste Manag 2021, 136, 266-72) has evaluated the application of microalgae in the treatment of winemaking lees digestate, however, only limited to the application of batch tests with high dilutions (1:5), yielding 1.36±0.08 g/l of microalgal biomass after 10 days. However, the process described by Scarponi et al., being batch-based, is not applicable on an industrial scale, which instead requires in-depth research and development of the optimal growth and effluent quality conditions in order to bring the process into continuous or semi-continuous mode.

The objective of the present invention is therefore to provide an industrial process for the semi-continuous/continuous cultivation of microalgae on anaerobic digestion wastewater, aimed at the complete recovery of the microalgae biomass and simultaneous purification of the effluent in order to be possibly reused (reduction of water consumption). The microalgae biomass can be exploited for the production of biodiesel and/or other high value-added by-products. Finally, the further objective of the present invention is to provide a process for the treatment and exploitation of winemaking lees that can be applied on site for small and large wineries.

### DEFINITIONS AND ABBREVIATIONS

BOD: Biological Oxygen Demand
COD: Chemical Oxygen Demand
CSTR: Continuous Stirred Tank Reactor
AD: Anaerobic Digestion
DIG: digestate from AD
HRT: Hydraulic Retention Time
OLR: Organic Load Rate
WL: White Lees
RL: Red Lees
PL: Pink Lees
WAS: Waste Activated Sludge
PBR: PhotoBioReactor
SGP: Specific Gas Production
SSC: Steady State Conditions

### SUMMARY OF THE INVENTION

The subject matter of the present invention is a process according to claim 1. The present invention solves the above-mentioned problems associated with microalgal cultivation on anaerobic digestion wastewater by means of a cultivation process of microalgal biomass comprising feeding the algal culture in semi-continuous without recirculation of the biomass with an undiluted centrifuged anaerobic co-digestate of any type of wine-making lees obtainable from wine production and activated sludge (WAS) from wine production water treatment.

Wine digestate can be applied for microalgal cultivation upon only solid-liquid separation (preferably centrifugation) to remove suspended solids, without sterilisation treatments and without dilution. According to the invention, microalgal cultivation on wine digestate is carried out in a semi-continuous mode without recirculation of the biomass, achieving the combined effect of phyto-depuration of the effluent and recovery of microalgae biomass rich in high value-added by-products.

The application of a coupled DA-microalgae system, such as the present invention, for the treatment of wine lees is surprisingly advantageous as an efficient and applicable treatment for wine producers on site. By applying the AD system to the wine lees treatment, the organic substrate is converted into biogas (a renewable energy source) and the effluent obtained (digestate) has chemical characteristics, such as a high nutrient content (N, P, C), that make it an ideal growth substrate for microalgae. The use of the digestate for the cultivation of microalgae has the dual advantage of phyto-depurating the digestate and of producing microalgal biomass that can find applications in a biorefinery context for the production of other products through green chemistry.

A final relevant aspect of the process of the invention is the absence of microalgal biomass recirculation, which is usually applied to treat high wastewater flow rates by reducing the risk of culture wash out. Biomass recirculation implies a limitation in the use of microalgae in wastewater treatment. In fact, recirculation requires a number of unitary operations in addition to the process that are necessary for the concentration of the biomass, negatively affecting both plant costs as well as revenue deriving from the biomass itself, thus limiting the entire treatment to phyto-purification alone.

The subject matter of the present invention is also a wine-growing wastewater treatment plant for carrying out the process according to the present invention, said plant according to claim 8.

### DETAILED DESCRIPTION OF THE INVENTION

The treatment process must take into account two main limiting factors: the concentration of inhibiting compounds (ammonia) and the species-specific cell regeneration time. For this reason, the microalgal species that can potentially be applied effectively for the digestate treatment in semi-continuous/continuous mode are Chlorella spp., Tetradesmus spp.

The batch experiment reported by Scarponi et al. (Waste Manag 2021, 136, 266-72) identifies the complete removal of ammonium in solution associated with a joint effect of i) ammonium stripping given by system aeration and ii) cell metabolism and growth times for the Chlorella vulgaris strain applied in batch cultivation. However, knowledge of the time required to remove contaminants (including agents inhibiting cell growth) and the time required to regenerate cell biomass in a batch cannot be directly transferred to a semi-continuous process. In fact, the effects of continuously feeding an undiluted digestate can lead to a possible accumulation of inhibiting compounds and a possible shortage of trace elements that cause a wash out of the culture, associated with a reduction in growth rate. For this reason, the definition of the correct growth conditions requires in-depth microalgal strain-specific evaluations and long-term experiments that are essential for validation, in order to achieve an efficient semi-continuous process for both biomass recovery and effluent treatment. As for the removal time and cell regeneration time, the choice of the type of reactor, the application of continuous or alternating lighting systems (with potential applications of different light wavelengths or applications of natural or artificial lighting) must be set depending on the species to be grown on wastewater for obtaining a semi-continuous/continuous treatment and for biomass recovery.

According to a preferred embodiment of the present invention, the strain applied for the cultivation in semi-continuous on wine co-digestate is Chlorella vulgaris.

According to the preferred embodiment of the present invention, the microalgal cultivation of Chlorella vulgaris in semi-continuous on the digestate was carried out in a vertical tubular PBR applying a lighting of 5-15 klux and an aeration of 3-4 l/min continuously; the applied artificial lighting was provided by 1-5 white neon lamps. The digestate applied for the cultivation in semi-continuous of microalgae according to the present invention can come from the anaerobic co-digestion treatment of WAS and any type of winemaking lees obtainable from the winemaking process. This digestate requires no sterilisation treatment and can be applied without dilution directly into semi-continuous/continuous systems for microalgal cultivation. The only required pre-treatment of the digestate is the removal of the solid particulate fraction, preferably by centrifugation (laboratory tested at 9000 rpm for 5 minutes).

The cultivation in semi-continuous of microalgal biomass on the digestate in photobioreactors involves feeding the digestate by applying specific flow rates into and out of the system that are equal (CSTR). The semi-continuous method consists in keeping the culture active for long periods of time by taking a predetermined percentage of the culture volume that will be replaced with the growth medium; there will be thereby a continuous supply of nutrients that will allow the algae to continue dividing, avoiding the senescence step. Specifically, the ratio of the total reactor volume to the incoming flow rate into the system (volume/time) defines the hydraulic retention time (HRT, time), i.e. the time required for the entire reactor volume to be replaced with the incoming medium. In case of anaerobic digestion, hydraulic retention times are generally between 20 and 30 days; in the case of wine wastewater, as reported by Da Ros et al. [Da Ros et al. Waste Manag 2014, 34, 2028-35] in the treatment of lees co-digestion and WAS, 23 days of HRT are optimal. In semi-continuous microalgae cultivation on municipal wastewater, HRTs typically range from 2 to 10 days (with recirculation of biomass), while treating more complex matrices such as digestate, HRTs of up to 20 days can be applied. Therefore, it is essential that the flow rate is defined in such a way that the concentration of biomass removed from the system is kept constant, guaranteeing growth times.

Preferably according to the present invention Chlorella vulgaris is cultivated with a HRT of 10-20 days, with a feeding 2-3 times a week of lees and centrifuged WAS co-digestate.

According to the preferred embodiment of the present invention, the application of 20-day HRT, with a feeding every other day (three times a week) of wine digestate is shown. The application of lower HRTs (10 days), for the purposes of the present invention, highlights the need for spacing the feeding operations (twice a week) in order to allow the proliferation of microalgal biomass.

Other important parameters to consider in the cultivation of microalgae with digestate are aeration and lighting, which are essential factors for maintaining the cultivation in a mixotrophic condition. While, on the one hand, the application of the liquid fraction of the undiluted digestate as a growth substrate containing high concentrations of ammonium in solution does not allow the proliferation of predators (i.e. rotifers), favouring the maintenance of the microalgae culture, the aeration of the system allows both mixing and a partial removal of the ammonium, with a reduction in treatment time. Ammonium accumulation in solution at concentrations >200 mg/l inhibits microalgal proliferation.

In the process, which is the subject matter of the present invention, microalgae biomass is not recirculated within the photobioreactor for digestate treatment. The biomass coming out of the photobioreactor can then be collected and applied for the production of high value-added secondary products in a variety of production sectors, from new green methods for producing energy from renewable sources to the recovery of high value-added secondary products for the cosmetic, feed and pharmaceutical industries.

The application of the coupled AD-microalgae system according to the present invention for the treatment on site of wine-making lees can be applied in the treatment processes as shown in FIG. 1, where the possibility of treating the flow of post-AD liquid digestate with a reduction of the ammonia nitrogen flow usually recirculated at the plant head is also identified. In addition, the AD process coupled with microalgal cultivation according to the present invention can also benefit from the possibility of biogas upgrading. Biogas upgrading means the removal of CO₂ present in biogas produced by AD systems to obtain a biogas with a methane content >95%. The biotransformation process of organic matter by the syntrophic microbial system of AD systems leads to the final production of biogas consisting of methane (60-70%) and carbon dioxide (30-40%). Carbon dioxide must therefore be removed in order to obtain bio-methane that can be introduced into the natural gas distribution network. The are several technologies of biogas upgrading and in most cases they are of the chemical-physical type; microalgae, being photosynthetic micro-organisms, are able to use inorganic carbon (CO₂) through the photosynthetic process by bubbling biogas in photobioreactors. Therefore, according to a preferred embodiment of the present invention, the biogas leaving the digester can be blown into PBRs in which algal biomass is cultivated to recover a biogas whose CO₂ content has been reduced.

The extraction of the lipid fraction from algal biomass for producing biodiesel implicitly also involves producing waste associated with cell residues that can further be applied as co-substrates for producing energy (biogas/ethanol) or converted to other applications from animal feed to the production of new materials. Preferably, the recovery of microalgal biomass according to the present invention takes place by applying a flocculation step prior to the recovery by solid-liquid separation (preferably centrifugation). The flocculation of microalgal biomass by the addition of organic flocculants (e.g. proyamine) or bio-flocculants (e.g. from algae, fungi or plants) brings about a reduction in the energy demand, and thus cost, associated with the downstream of the process. The application of flocculation results in an energy demand of 11.97 MJ kg⁻¹, which is lower than the 13.8 MJ kg⁻¹ that are required for biomass recovery in the absence of pre-flocculation. Preferably according to the present invention the effluent from PBR is recycled into further PBR. This recirculation system implies a further reduction in the costs associated with cultivation and an increase in the production of biomass rich in high-added-value secondary products, such as proteins, lipids and starch, which would find application in the circular economy.

It is therefore also a subject matter of the present invention is also a wine-growing wastewater treatment plant wherein, preferably, said at least one PBR has a second inlet in fluid communication with the first outlet of the AD to send the biogas leaving the AD to said at least one PBR.

Preferably, the plant of the invention further comprises a second solid-liquid separation system having an inlet in fluid communication with the outlet of said at least one PBR, a first outlet for collecting the solid biomass and a second outlet for collecting the liquid fraction of the culture broth, said second outlet in fluid communication with the head of the wine-growing wastewater treatment plant.

The present invention can be better understood in light of the following embodiments.

### BRIEF DESCRIPTION OF THE DRAWINGS

FIG. 1 shows the application of the treatment in semi-continuous of the coupled AD-microalgae system according to the present invention in a wine-growing wastewater treatment system. The dotted lines inserted in the treatment process indicate the process variations according to the present invention in a wine-growing wastewater treatment system i) the post-AD liquid fraction is sent to the microalgal cultivation as a growth medium; ii) the post-AD gaseous fraction is sent to the microalgal cultivation to obtain biomethane upgrading; iii) the post-microalgal cultivation liquid fraction can be recirculated in the plant head, iv) the biomass obtained from the microalgal cultivation can undergo treatments to extract starch, proteins, pigments and lipids; v) the post-extraction cell residues can be sent as a co-substrate in the AD treatment system for the biogas production increase.

FIG. 2 shows the monitoring of the AD system fed with white lees (WL), red lees (RL) and pink lees (PL) according to the example 1. a) monitoring of partial and total alkalinity and pH; b) monitoring of OLR and specific gas production (SGP).

FIG. 3 shows the monitoring of dry weight and cell density of the microalgae culture fed semi-continuously with wine digestate without biomass recirculation by applying 20-day HRT according to example 2.

FIG. 4 shows the monitoring of dry weight and cell density of the microalgae culture fed semi-continuously with wine digestate without biomass recirculation by applying 10-day HRT with feeding every other day according to example 2.

FIG. 5 shows the monitoring of dry weight and cell density of the microalgae culture fed semi-continuously with wine digestate without biomass recirculation by applying 10-day HRT with C. vulgaris, with two weekly feeding operations according to example 2.

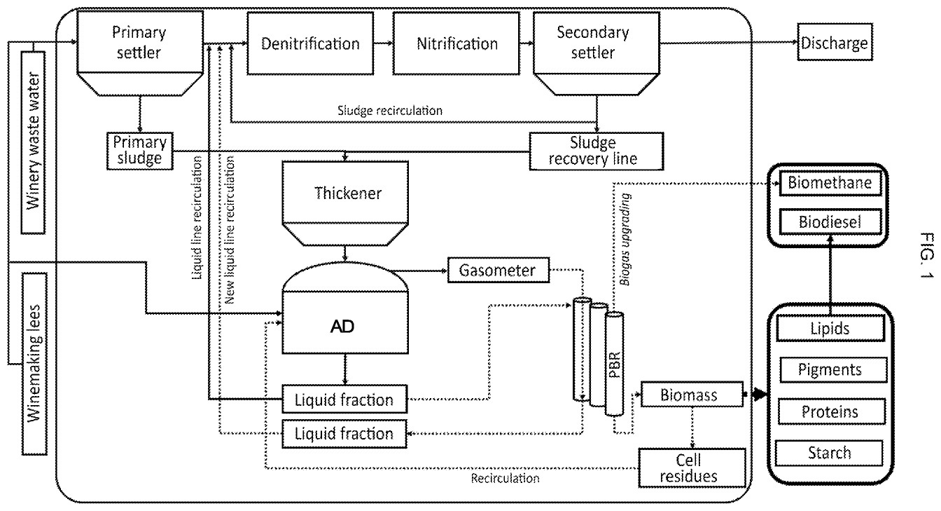

### EXPERIMENTAL PART

### Example 1—Co-Digestion Treatment of Winemaking Lees and Waste Activated Sludge

The AD system for winemaking lees treatment has been monitored for more than three years continuously by applying, as co-digestion feeding, activated sludge from the winery water treatment (WAS) and all types of wine-making lees obtainable from wine production: white lees (WL), red lees (RL) and a mix of the two (pink lees, PL).

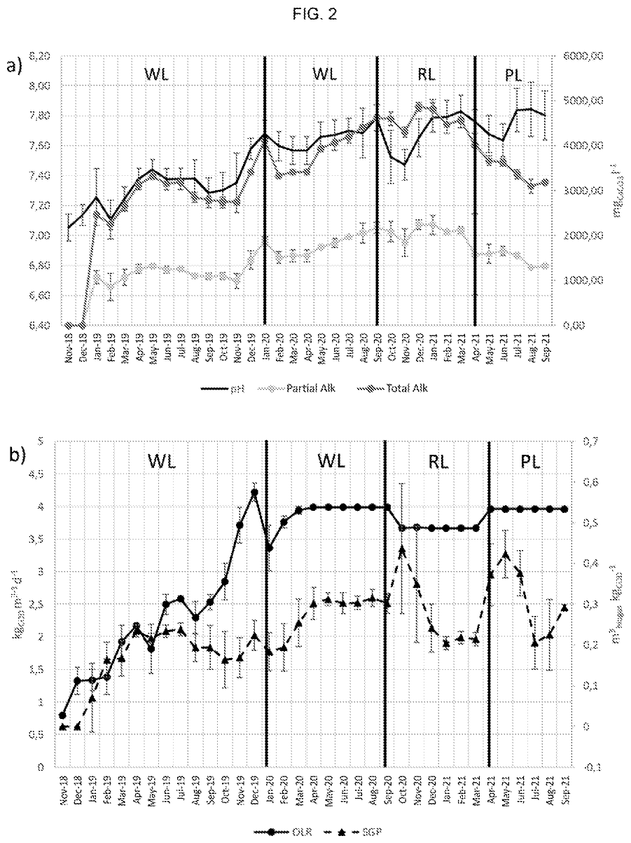

The AD treatment was applied in a 4-litre pilot-scale reactor with a 20-day HRT and organic loading rate (OLR) of 3.77±0.36 gCOD/(l*d), at a stable temperature of 37° C. During steady-state conditions (SSC) of feeding at WL, RL and PL, the digestate (DIG-WL, DIG-RL and DIG-PL) was collected, characterised and used for microalgal cultivation in semi-continuous mode after only centrifugation at 9000 rpm for 5 minutes for removal of suspended solids. In no case was sterilisation of the digestate carried out. The change of feeding between the different wine-making lees was carried out by direct switches. As reported in FIG. 2, the change of lees for feeding the DA system in co-digestion did not lead to changes in pH and fatty acid accumulation in the digestate, in terms of digestate composition, the greatest effects at the change of feeding were found for sCOD in solution and in biogas production.

The AD system for WAS and lees co-digestion was stable over the three years of monitoring, where a stable methane production of 63% was identified for all lees applied to the system. As reported in Table 1, the application of RL as a co-substrate affects the biogas production rate (GPR) and the elevation of sCOD in solution. This effect of lowering GPR and raising sCOD may be associated with the increased presence of polyphenols in RL that may have partially inhibited the micro-organisms in the AD system. The characterisations of the different types of digestate obtained from the treatment of the different wine-growing lees (Table 1) are comparable both with each other and with the work in the literature for the treatment of wine-growing lees on a pilot scale [Da Ros C, et al. J Environ Manage 2017, 203, 745-52]

TABLE 1
Characterisation of DIG-WL, DIG-RL and DIG-PL during steady-state conditions (SSC).

| | | | | Da Ros. et al. |
|---|---|---|---|---|
| | DIG-WL | DIG-RL | DIG-PL | 2017 |
| TS (gTS/kg) | 34.89 ± 1.74 | 38.09 ± 2.45 | 35.93 ± 4.47 | 24.30 ± 2.90 |
| TVS (gTVS/kg) | 15.03 ± 1.12 | 17.90 ± 2.47 | 14.76 ± 2.01 | 14.20 ± 1.70 |
| Ammonia N (gN-NH₄⁺/l) | 0.84 ± 0.01 | 0.35 ± 0.07 | 0.36 ± 0.13 | 0.40 ± 0.05 |
| Phosphates (mg/l) | 266.34 ± 19.31 | 161.65 ± 0.21 | 372.19 ± 12.02 | 213.84 ± 38.88 |
| TKN (mgN/gTS) | — | — | — | 36.30 ± 4.50 |
| COD on dry weight (gO₂/gTS) | 610.41 ± 10.59 | 868.05 ± 39.65 | — | 559.67 |
| sCOD (gO₂/l) | 0.23 ± 0.04 | 0.41 ± 0.03 | 0.25 ± 0.04 | 0.40 ± 0.20 |
| VFA tot (gCOD/l) | 0.14 ± 0.05 | — | — | — |
| pH | 7.69 ± 0.01 | 7.80 ± 0.02 | 7.78 ± 0.14 | 7.46 ± 0.19 |
| Total alkalinity (g CaCO₃/I) | 2.27 ± 0.07 | 2.47 ± 0.09 | 1.85 ± 0.07 | 2.24 ± 0.2 |
| Partial alkalinity (g CaCO₃/l) | 1.95 ± 0.11 | 2.14 ± 0.08 | 1.49 ± 0.17 | 1.37 ± 0.12 |
| OLR (kg_{COD}/m⁻³ *d) | 3.99 | 3.66 | 3.96 | 3.2 |
| GPR (m³/m³*d) | 1.23 ± 0.02 | 0.77 ± 0.02 | 0.85 ± 0.05 | 1.2 |
| SGP (m³_{biogas}/kg_{COD}) | 0.30 ± 0.00 | 0.21 ± 0.00 | 0.21 ± 0.01 | 0.38 ± 0.04 |

### Example 2—Cultivation in Semi-Continuous of Microalgal Biomass on the Digestate without Biomass Recirculation

The cultivation in semi-continuous without recirculation of the microalgal biomass was studied by applying Chlorella vulgaris as the microalgal strain for the case study.

To confirm the need for optimisation of feeding times in order to achieve an efficient and stable treatment in the long term, tests were carried out by evaluating the effect of two different HRTs (20 days and 10 days) by feeding every other day or by increasing the spacing between feeding operations with the wine digestate. Recirculation of microalgal biomass was not applied in all experiments. The digestate applied for the cultivation in semi-continuous was submitted to centrifugation at 9000 rpm for 5 minutes to remove the solid fraction in the medium as the only pre-treatment. No sterilisation and dilution treatment was applied to the digestate. The tests were carried out on 4-litre vertical tubular PBRs (47 cm×12 cm). Lighting was provided by four white neon lamps for each reactor (10.17±1.12 klux) and areation (3.4 l min⁻¹, Amtra Technik Mouse 4, Germany) in a continuous mode. The initial dry weight value of the inoculum was 0.70±0.08 g l⁻¹ and cell count analysis was carried out daily to identify morphological changes, potential contamination (i.e. rotifers) and cell density monitoring during the tests. Each test in semi-continuous cultivation was monitored until the end of the second/third HRT cycle. Each test was carried out in duplicate.

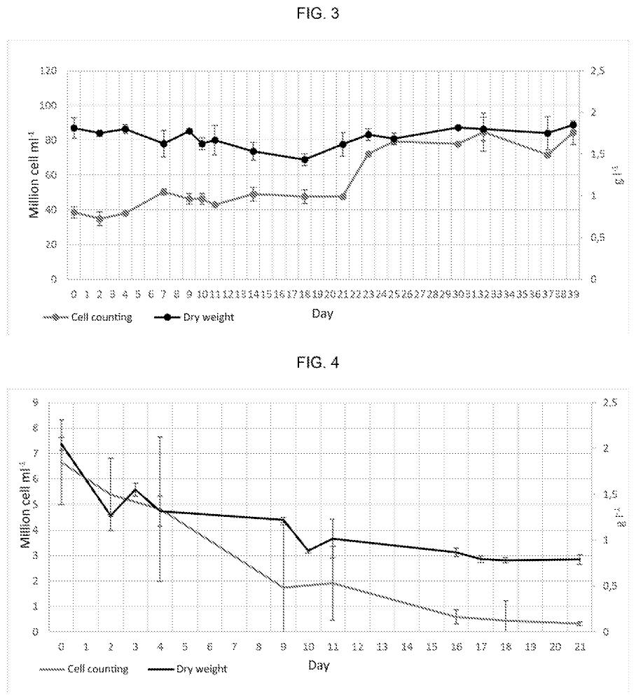

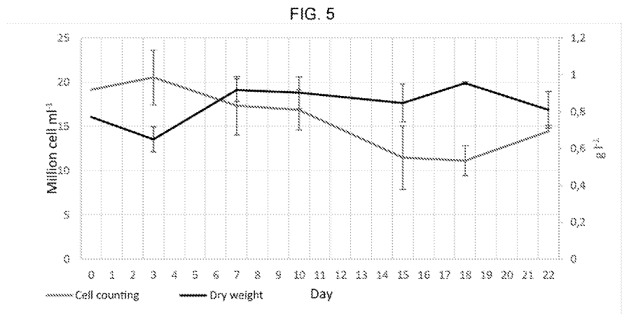

Feeding wine digestate every other day by applying a 20-day HRT for the cultivation of C. vulgaris without recirculation of the biomass resulted in a stable biomass concentration of 1.80±0.05 g l⁻¹ (FIG. 3). In contrast, the application of 10-day HRT with a feeding every other day showed a reduction in the concentration of microalgae biomass due to wash out, down to about 1 g/l (FIG. 4). As for chromatographic analysis, the reduction of ammonium concentration in solution during the test was 30%, reaching a concentration above 200 mg/l, a value that inhibited microalgal proliferation. The test confirmed that in order to achieve an efficient treatment of the digestate by microalgal cultivation in semi-continuous, there is a need to increase the spacing of the photobioreactor feeding with the wine digestate, thus allowing the proliferation of the biomass of Chlorella vulgaris and the removal of ammonium in solution. To date, the process is being verified by applying a 10-day HRT with C. vulgaris, with two weekly feeding operations without recirculation of the microalgal biomass (FIG. 5). Preliminary data show that the microalgal biomass concentration is stable at around 1 g/l with 95% reduction in ammonium concentration compared to digestate values. In order to reduce HRT, it is therefore necessary to consider the cell growth rate of the microalgal species and the effectiveness of ammoniacal nitrogen content reduction to avoid accumulation and inhibition of proliferation. (FIG. 3 does not report the start-up step of the system).

The characterisation of the microalgal biomass obtained during the treatment in semi-continuous by applying a 20-day HRT is shown in Table 2. The results obtained for the amount of oil accumulated in the microalgal biomass by applying a cultivation in semi-continuous without recirculation of the microalgal biomass on wine digestate underline the maximum lipid production that can be accumulated by the strain when compared to its batch cultivation on wine digestate [Scarponi P, et al Waste Manag 2021, 136, 266-72].

TABLE 2
Characterisation of the biomass and microalgal lipid fraction of
C. vulgaris leaving the cultivation in semi-continuous system
without recirculation of biomass on digestate from winemaking lees.

| HRT | 20 days |
|---|---|
| Lipids (%) | 33.48 ± 7.54 |
| Starch (%) | 5.22 ± 1.08 |
| Proteins (%) | 57.85 ± 10.14 |
| Ch a (mg g_{biomass}⁻¹) | 4.99 ± 0.52 |
| Ch b (mg g_{biomass}⁻¹) | 1.45 ± 0.10 |
| Total carotenoids (mg g_{biomass}⁻¹) | 1.71 ± 0.24 |
| Myristic acid C14:0 (% w/w) | 2.29 ± 0.77 |
| Palmitic acid C16:0 (% w/w) | 39.20 ± 1.97 |
| Stearic acid C18:0 (% w/w) | 23.61 ± 8.30 |
| Palmitoleic acid C16:1 (% w/w) | 0.93 ± 0.22 |
| Oleic acid C18:1 (% w/w) | 11.58 ± 5.93 |
| C16:2 (% w/w) | 3.32 ± 3.35 |
| Linoleic acid C18:2 (% w/w) | 15.96 ± 13.19 |
| Linolenic acid α-18:3 (% w/w) | 0.5 ± 0.70 |

The characterisation of the lipid fraction identifies the potential direct application of the microalgal oil for the production of high-quality biodiesel pursuant to EN14214, which specifies that the linolenic acid concentration must be lower than 12% w/w for direct application of the extracted oil for the production of high-quality biofuel.

The preliminary assessment for the application of extracted oil for biofuel production was carried out based on the model reported by Giakoumis et al. (Fuel 2018, 222, 574-85) that identifies the equations needed to evaluate cetane number (CN), viscosity, calorific value and density based on the composition of the extracted oil. As reported in Table 3, the values obtained for the type of oils present in the biomass obtained from the experiment are in line with the values obtained by Giakoumis et al. (2018) for high quality biodiesel referring to the limits reported in EN14214. Table 3: Values of cetane number (CN), viscosity, higher and lower heating value (HHV and LHV) and density based on the composition of the oil extracted from the cultivation of C. vulgaris on digestate from winemaking lees without recirculation of microalgae biomass.

| | | Average values based |
|---|---|---|
| HRT | 20 days | on Giakoumis et al. |
| CN | 64.27 | 54.90 |
| Density (ρ, kg m⁻³) | 873.15 | 880.24 |
| Viscosity (δ, mm² s⁻¹) | 5.22 | 4.18 |
| HHV (kJ kg⁻¹) | 40,504.38 | 40,136.74 |
| LHV (kJ kg⁻¹) | 37,025.04 | 37,649.09 |

The characterisation of the output from PBR was carried out with a view to assessing the phyto-depurative effect of the digestate carried out by microalgae. The total removal of ammonium in solution was highlighted during the 20-day HRT treatment. The effluent resulting from the treatment with microalgae does not comply with the limits for discharge into the sewerage system and surface water (D. Lgs. 152/06); recirculation of this flow in the wastewater treatment plant head is, however, advantageous considering the absence of ammoniacal nitrogen compared to the post AD recirculation flow, but with the presence of its oxidised forms, which therefore favours a reduction in aeration consumption. The characterisation of the sCOD contained in the effluent is being verified in order to determine the presence of exopolysaccharides secreted by the microalgae.

1. A process of cultivation of a microalgae biomass in one or more photobioreactors (PBR) said process comprising feeding the algal culture in semi-continuous without recirculation of the biomass, said feeding with the undiluted liquid fraction effluent from an anaerobic co-digestion (AD) of any type of winemaking lees obtainable from a wine production and activated sludge from a water treatment of a wine production (WAS).

2. The process according to claim 1 wherein the hydraulic retention time (HRT) of the biomass in the PBRs is 10-20 days and the feeding with the liquid fraction of digestate takes place every 2-3 days.

3. The process according to claim 1 wherein the biogas produced by the anaerobic co-digestion is bubbled into the PBRs to offer nutrients to the algal culture and simultaneously obtain upgrading of the biogas to biomethane.

4. The process according to claim 1 wherein the culture broth exiting from the PBR is subjected to solid-liquid separation for the recovery of the biomass which is then subjected to starch and/or protein and/or pigments and/or lipids extraction processes and post-extraction cell residues are sent as co-substrate in anaerobic co-digestion (AD) to increase biogas production.

5. The process according to claim 1 wherein the biomass exiting from the PBRs is subjected to flocculation by adding at least one organic flocculant before being subjected to solid-liquid separation for the recovery of the biomass.

6. The process according to claim 1 wherein the post-cultivation algal liquid fraction is recirculated at the head of a wine-growing wastewater treatment plant.

7. The process according to claim 1 wherein the algal biomass is Chlorella Vulgaris or Tetradesmus spp.

8. A wine waste treatment plant for carrying out the process according to claim 1, said plant comprising:
an anaerobic digester (AD) for the anaerobic co-digestion of winemaking lees and activated sludge from the water treatment of wine production (WAS), said AD having a first inlet for the entry of the winemaking lees, a second inlet for the WAS inlet, a first outlet for the collection of biogas produced by anaerobic co-digestion and a second outlet for the digestate;
a first liquid-solid separation system having an entry in fluid communication with the second outlet of the digester and an outlet for the liquid fraction of the digestate;
at least one PBR for the cultivation of microalgae biomass, called PBR having an inlet in fluid communication with the outlet of the solid-liquid separation system and an outlet for the culture broth.

9. The plant according to claim 8 wherein said at least one PBR has a second inlet in fluid communication with the first outlet of the AD to send the biogas leaving the AD to said at least one PBR.

10. The plant according to claim 8 comprising a second solid-liquid separation system having an inlet in fluid communication with the outlet of said at least one PBR, a first outlet for collecting the solid biomass and a second outlet for the collection of the liquid fraction of the culture broth, said second outlet in fluid communication with the head of the wine waste treatment plant.
